(19) United States
(12) Patent Application Publication
(10) Pub. No.: US 2004/0157925 A1
(43) Pub. Date: Aug. 12, 2004

(54) Stable pharmaceutical composition of pravastatin

(76) Inventors: Vishnubhotla Nagaprasad, Hyderabad (IN); Rajiv Malik, Delhi (IN)

Correspondence Address: Jayadeep R Deshmukh, Ranbaxy Pharmaceuticals Inc, Suite 2100, 600 College Road East, Princeton, NJ, 08540, US

(21) Appl. No.: 10/473,208

(22) PCT Filed:
(86) PCT No.: PCT/IB02/00882

(30) Foreign Application Priority Data
Mar. 27, 2001 (ZA) 375/DEL/01

(51) Int. Cl. A61K 31/22; A61K 9/20

(52) U.S. Cl. 514/549; 424/465

(57) ABSTRACT

The present invention relates to a stable pharmaceutical composition comprising pravastatin or its pharmaceutically acceptable salts and a carrier, which imparts a pH between 6.5 and 8.5 to an aqueous dispersion of said composition. The invention also relates to a process for making the pharmaceutical composition.

### FIELD OF THE INVENTION

The present invention relates to a stable pharmaceutical composition comprising pravastatin or its pharmaceutically acceptable salts and a carrier, which imparts a pH between 6.5 and 8.5 to an aqueous dispersion of said composition. The invention also relates to a process for making the pharmaceutical composition.

### BACKGROUND OF THE INVENTION

Pravastatin, chemically known as (+)-(3R, 5R)-3,5-dihydroxy-7-[(1 S,2S,6S, 8S, 8aR)-6-hydroxy-2-methyl-8-[(S)-2-methylbutyryloxy]-1,2,6,7,8,8a-hexahydro-1-naphthyl]heptanoate, and its pharmaceutically acceptable salts has been described in U.S. Pat. No. 4,346,227 which is incorporated herein by reference.

Pravastatin is an HMG-CoA reductase inhibitor which reduces plasma cholesterol levels by inhibiting de novo cholesterol synthesis and increasing the receptor mediated catabolism of low density lipoproteins. The drug exhibits hepatocellular tissue selectivity, with greatest inhibition of cholesterol synthesis occurring in the liver and thereby inhibiting the unwarranted effects on cholesterol synthesis in nonhepatic (peripheral) cells. Its favourable effects on cardiovascular morbidity and total mortality renders it as an effective alternative to currently used HMG-CoA reductase inhibitors for patients with elevated cholesterol levels, multiple risk factors or coronary heart disease.

The therapeutic efficacy of any drug depends to a considerable extent on the design of its pharmaceutical formulation. The physico-chemical attributes and bio-pharmacological characteristics account for the formulation of a stable and bioavailable pharmaceutical composition.

Pravastatin sodium is relatively polar and hydrophilic in nature. It is susceptible to heat, light and moisture. It is also sensitive to a low pH environment and is very unstable at pH 3 or less as found in the stomach wherein the hydroxy acids degrade to form lactone and an inactive isomer primarily, 3-a-hydroxy-isopravastatin (Triscari et. al., J. Clin. Pharmacol, 1995; 35:142). The acid instability of pravastatin reduces its bioavailability and results in degradation of pravastatin following oral administration.

The literature discloses various approaches to obviate problems related to unfavourable absorption characteristics of pravastatin due to acid sensitivity.

One such approach mentioned in the prior art pertains to the use of agents that are basic in nature and impart alkaline pH. EP 336,298, for example, describes a stabilized pharmaceutical composition of pravastatin comprising drug, fillers, binders, disintegrants, lubricants and basifying agents to impart a desired pH of at least 9 and preferably about 10 to an aqueous dispersion of said composition. The essence of the invention is to maintain an alkaline environment to combat the low pH sensitivity of the drug. While such an approach may be suitable for enhancing the stability of the drug, however, the local alkaline environment occurring at the site of dissolution of the composition may damage the natural acidic mantle of the gastric mucosa especially, in chronic therapies with HMG-CoA reductase inhibitors.

Other techniques which have been described in the prior art for enhancing the stability of pravastatin include the formulation of “inclusion compounds” by their complexation with agents such as cyclodextrins. WO 99/49896 discloses a composition of sodium pravastatin characterized in that the composition contains β-cyclodextrin as a stabilizer. Cyclodextrin surrounds the drug molecules and prevents its explosure to the acidic environment. As stated and exemplified in the specification, the amount of β-cyclodextrin is advantageously used in the range of 50-5000 weight parts in proportion to 100 weight parts of sodium pravastatin, below which, the drug is insufficiently stabilized and degrades at high humidity and temperature. It is well recognized by those skilled in the art that the desired stability may be achieved by application of such an approach but not without compromising the release of the drug.

Still other techniques are directed towards use of protective coatings to prevent release of pravastatin in the stomach. U.S. Pat. No. 5,225,202 discloses an enteric coated pharmaceutical composition of pravastatin in the form of tablet, beadlet, pellet or particle that is enteric coated with neutralized hydroxypropylmethyl cellulose phthalate and a plasticizer which affords protection in a low pH environment of 3 or less while release medicament at a pH of 4.5 or higher. It is well known to the formulation scientist that phthalate polymers are prone to hydrolysis. Also, due to aging, the properties of the polymer change which could have significant effect on both ultimate dissolution behaviour and mechanical properties of the applied coating. Further, with time, under ambient conditions, the enteric coating gives an acidic residue which may degrade pravastatin within the formulation itself. Furthermore, an enteric coated formulation requires prolonged time to attain the effective serum concentration. Additionally, the application of the enteric coating is an additional operation which increases the length of the manufacturing process and thereby the cost of the product.

As aforementioned, several pharmaceutical compositions have been described which relate to the means to improve the stability, absorption and thus bioavailability profile of pravastatin. However, none of the solutions described above are completely satisfactory.

As aforesaid, one of the requirements for an acceptable pharmaceutical composition is that it must be sufficiently stable so as not to exhibit substantial decomposition of the active ingredient during the time between manufacture of the composition and absorption of the drug in the body.

In light of the foregoing, the primary object of the present invention is to provide a process for the preparation of a pharmaceutical composition of pravastatin which is stable upon prolonged storage and provides the desired therapeutic effect while avoiding the heretofore mentioned disadvantages.

### SUMMARY OF THE INVENTION

It is an object of the present invention to provide a pharmaceutical composition of pravastatin which effects better stability and readier bioavailability. More particularly, the present invention provides a pharmaceutical composition which is stable and suitable for oral administration, comprising an effective amount of pravastatin or its pharmaceutically acceptable salts and a carrier, said carrier comprising at least one diluent and at least one lubricant to impart a pH between 6.5 and 8.5 to an aqueous dispersion of said composition.

According to another aspect of the present invention, it provides a stable formulation of pravastatin in the form of beads, pellets, granules, tablets and capsules, comprising pravastatin or its pharmaceutically acceptable salts and a carrier wherein the carrier comprises pharmaceutical adjuvants such as inert diluent, binder, glidant, anti-adherent, and the like. Also, the pharmaceutical composition in solid dosage form may be optionally coated with a rapidly dissolving water soluble polymer film coat.

The present invention is directed to a pharmaceutical composition exhibiting enhanced stability and bioavailability of pravastatin or its pharmaceutically acceptable salts which is attained through the use of processing techniques and adjuvants that do not adversely affect the stability of the drug.

Insofar as the techniques of formulation of pharmaceutical composition are concerned, two preparative routes are known, which are wet granulation and dry process which includes dry granulation (compaction or slugging) and direct compression. The comparative experimentation revealed that wet granulation of pravastatin resulted in deleterious effect on the stability of formulation. A dry processing technique instead, stabilized the formulation remarkably.

The present invention also provides a dry process for the preparation of pharmaceutical composition which is stable and suitable for oral administration, comprising mixing an effective amount of pravastatin or its pharmaceutically acceptable salts and a carrier, said carrier comprising at least one diluent and at least one lubricant to impart a pH between 6.5 and 8.5 to an aqueous dispersion of said composition.

Also, in the course of developing the present invention, many different pharmaceutical compositions containing pravastatin were formulated and tested for stability. Since this hydroxy acid compound (pravastatin) is susceptible to degradation to the lactone form in an acidic environment, it was necessary to stabilize its structural integrity in pharmaceutical formulations. Through extensive comparative experiments using various combinations of excipients, it was found that the use of sodium stearyl fumarate aided in stabilizing the formulation. The total related substances and the lactone formation was much lower when sodium stearyl fumarate was used as the lubricant.

### DETAILED DESCRIPTION OF THE INVENTION

According to the present invention, the pharmaceutical composition may contain one or more of a water soluble and/or water dispersible diluent as a granulation substrate or filler. Examples of water soluble diluents that may be used in the present invention include, but are not limited, to lactose, sucrose, calcium carbonate, calcium phosphate, calcium hydrogen phosphate, tribasic calcium phosphate, compressible sugar, calcium sulphate, sorbitol, mannitol, and other polyols, dextrates, dextrin, dextrose, maltodextrin, and the like. Water dispersible diluents which refer to water insoluble pharmaceutical excipients that disperse readily in water include but are not limited to cellulose based excipients such as microcrystalline cellulose, powdered cellulose, starches such as corn starch, pregelatinized starch, clays or clay minerals such as kaolin, bentonite, attapulgite, and the like.

In preferred embodiments of the present invention, the pharmaceutical composition contains calcium carbonate as the diluent.

The amount of diluent relative to the drug may vary depending on the nature of diluent, their physiochemical characteristics, total weight of the formulation, and other adjuvants that may be present as the integral part of the formulation. However, the diluent may be present in an amount from about 5% to about 95% by weight, more preferably from about 15% to about 80% by weight of the total weight of the pharmaceutical composition.

In accordance with this invention, the pharmaceutical composition may contain a lubricant for preventing sticking to metal tooling and easy flowability of the powder blend. The lubricants which are amenable to the pharmaceutical composition of the present invention include any of those that do not adversely affect the stability or pharmaceutical efficacy of the formulation. The lubricant selected should be such that there is no interaction which would substantially reduce the shelf life of the composition of the present invention. Examples of lubricants suitable for this invention include the lubricants well known in the pharmaceutical art such as sodium stearyl fumarate, palmitic acid, calcium stearate, magnesium stearate, talc, carnuba wax, zinc stearate, silicon dioxide, hydrogenated vegetable oil and the like.

In preferred embodiment of the present invention, the pharmaceutical composition contains sodium stearyl fumarate as the lubricant.

The composition of the invention may contain a lubricant in an amount from about 0.1% to about 15% and preferably from about 0.2% to about 10% by weight of the total weight of the composition.

Other possible and supplemental adjuvants such as binders, disintegrants, surface active agents, glidants, anti-adherents, colourants, and the like known as conventional by those skilled in the art may be included, optionally, in the inventive formulation. The present invention is not to be construed as being limited to any particular excipient or class of pharmaceutical excipients. Pharmaceutical adjuvants used must be of high purity and low toxicity to render them suitable for incorporation and administration thereof. The choices of these adjuvants and the amounts to be used are considered to be within the purview of one skilled in the art and would depend on the type of dosage form.

The pharmaceutical composition may contain a binder so as to form a cohesive mass of the powder blend. It may be any pharmaceutically acceptable, non-toxic, water soluble and/or water insoluble agent showering binding properties. The composition may contain a binder selected from among several applicable substances such as corn starch, polyvinyl alcohol, microcrystalline cellulose, polyvinylpyrrolidone, modified corn starch, sugars, gums, methyl cellulose, hydroxypropyl cellulose, and the like.

The requisite amount of binding agent used in this invention is an amount needed to obtain a cohesive mass of desirable strength that allows for the formation of granules or tablets of optimum hardness. The binding agent may be present in an amount from about 0.1% to about 10% by weight and preferably from about 0.25% to about 7.5% by weight of the composition.

According to the present invention the composition may contain a disintegrating agent. Suitable disintegrating agents that can be used in the present invention include starch, croscarmellose sodium, sodium starch glycolate, crospovidone, cross-linked carboxymethyl starch, magnesium aluminium silicate, polyacrylin potassium and the like. The disintegrating agent may be present in an amount from about 1% to about 10%, preferably from about 2% to about 7% by weight of the total weight of the composition.

In accordance with this invention, the pharmaceutical composition may contain a surface active agent to facilitate the wettability and dissolution of the drug. The surfactant used may be selected from those conventionally used in pharmaceutical preparations such as sodium lauryl sulphate, polyoxyethylene-polyoxypropylene copolymers (poloxamer), polysorbates (such as available as Tween 20, Tween 40, Tween 60 and the like) and the like.

The composition of the invention may contain surface active agent in an amount from about 1% to about 5% and preferably from about 1.5% to about 3.5% by weight of the total weight of the composition.

In addition to the above ingredients, colloidal silicon dioxide, and the like as glidants, talc, and the like as an anti-adherent and iron oxides, and the like as colorants may be incorporated into the carrier.

The present invention is not to be construed as being limited to any particular excipient or class of pharmaceutical excipients. The choice of adjuvants and the amounts to be used are considered to be within the purview of one skilled in the art. The amount of pharmaceutical adjuvants should be such that the aqueous dispersion of the said composition imparts a pH between 6.5 and 8.5.

According to the present invention the pharmaceutical composition may be prepared either in the form of pellets, beads, granules, tablets and capsules.

The pharmaceutical composition in accordance to the present invention may be optionally coated with rapidly dissolving water soluble film coat. The examples of water soluble polymers include hydroxypropyl methylcellulose, hydroxypropyl cellulose and the like. The solid unit dosage form in accordance with the present invention may be coated to a weight build up of about 1% to about 10% by weight, preferably from about 1% to about 4% by weight of the total weight of the composition.

According to the present invention wherein the pharmaceutical composition is in the form of capsule dosage form, the capsule shell may be of a hard gelatin or a soft gelatin type. Furthermore, capsules made of starch or hydroxypropyl methylcellulose may also be used.

Stability studies for the different pharmaceutical compositions were performed using the technique known as accelerated stability testing. In such studies, samples were stored at the conditions of elevated temperature and high humidity (40° C./75% RH). At the end of the desired time schedule, the samples were analyzed for the drug content and total related substances (degradation products) using high performance liquid chromatographic techniques (HPLC).

According to the present invention, the pharmaceutical composition is prepared by blending pravastatin sodium with carrier comprising at least one diluent and at least one lubricant and the optionally added adjuvants including anti-adherents and glidants. The blend is directly compressed into tablets or may be filled into capsules.

Alternatively, the pharmaceutical composition is prepared by blending the aforementioned ingredients with only a portion of the lubricant. The blend is roll compacted and then sized to obtain granules. The granules may be filled into capsules or compressed into tablets.

In those embodiments of the present invention wherein the foregoing composition is in the form of spherical pellets or beads, the art of producing such dosage forms by extrusion and spheronisation techniques or techniques based on high shear granulation or fluidized bed techniques may be used. Single unit pellets can be produced on industrial scale using lozenge and troches cutting machines.

The following examples further illustrate this invention and are not to be construed as limiting the scope but read in conjunction with the description above, provide further understanding of the present invention and an outline of the process for preparing the composition of the invention.

### EXAMPLE 1

This example illustrates the present invention in the form of tablets using a dry process for preparation and sodium stearyl fumarate as the lubricant having a composition as given in Table 1.

TABLE 1

| Ingredients | % w/w |
|---|---|
| Pravastatin sodium | 10 |
| Lactose, anhydrous | 75.5 |
| Calcium carbonate & Maltodextrin | 12.5 |
| (Calcarb 4450 PG) | |
| Sodium stearyl fumarate | 2.0 |

Pravastatin sodium, lactose, calcium carbonate and maltodextrin and sodium stearyl fumarate were blended together and sifted through a sieve of 355 μm mesh (British Standard Sieve (BSS) No. 44). The blend was directly compressed into tablets.

The compressed tablets were packed in an aluminium strip pack and stored at 40° C. and 75% RH. A stability indicating assay procedure was used to determine the drug content and the total related substances. The results are recorded in Table 2.

TABLE 2

Time

| Stability Parameters | Initial | 3 Months |
|---|---|---|
| Assay | 106.5% | 106.5% |
| Total related substances | 0.409% | 1.271% |
| pH of the aqueous | 8.27 | 8.21 |
| dispersion | | |

The results indicate that even after 3 months there was no significant difference in assay, total related substances or pH of the aqueous dispersion of the formulation.

### EXAMPLE 2

This example illustrates the present invention in the form of tablets with croscarmellose sodium as the disintegrating agent. The pharmaceutical composition is given in Table 3.

TABLE 3

| Ingredients | % w/w |
|---|---|
| Pravastatin sodium | 13.3 |
| Lactose, anhydrous | 64.0 |
| Croscarmellose sodium | 4.0 |
| Calcium carbonate & Maltodextrin | 16.7 |
| (Calcarb 4450 PG) | |
| Sodium stearyl fumarate | 2.0 |

The tablets were prepared and packed as described in Example 1. The tablets were characterized for stability as disclosed in Example 1 and the results are tabulated in Table 4.

TABLE 4

Time

| Stability Parameters | Initial | 3 Months |
|---|---|---|
| Assay | 99.8% | 98.5% |
| Total related substances | 0.523% | 1.250% |
| pH of the aqueous | 8.22 | 8.20 |
| dispersion | | |

The results indicate that even after 3 months there was no significant difference in the values.

### Example 3

This example illustrates the present invention in the form of tablets using a dry process for preparation having a composition as given in Table 5.

TABLE 5

| Ingredients | % w/w |
|---|---|
| Pravastatin sodium | 13.33 |
| Lactose, anhydrous | 84.67 |
| Sodium stearyl fumarate | 2.0 |

Pravastatin sodium, lactose and sodium stearyl fumarate were blended together and sifted through a sieve of 355 μm mesh (British Standard Sieve (BSS) No. 44). The blend was directly compressed into tablets.

The compressed tablets were packed in High Density Polyethylene (HDPE) bottles which were induction sealed and stored at 40° C. and 75% RH. A stability indicating assay procedure was used to determine the drug content and the total related substances. The results are recorded in Table 6.

TABLE 6

Time

| Stability Parameters | Initial | 3 Months |
|---|---|---|
| Assay | 95.3% | 95.8% |
| Total related substances | 0.572% | 0.678% |
| pH of the aqueous | 7.38 | 7.37 |
| dispersion | | |

The results indicate that even after 3 months there was no significant difference in assay, total related substances or pH of the aqueous dispersion of the formulation.

While this invention has been described with an emphasis upon preferred embodiments, it will be obvious to those of ordinary skill in the art that variations in the preferred methods of the present invention may be used and that it is intended that the invention may be practiced otherwise than as specifically described herein. Accordingly, this invention includes all modifications encompassed within the spirit and scope of the invention as defined by the following claims.

While the present invention has been described in terms of its specific embodiments, certain modifications and equivalents will be apparent to those skilled in the art and are intended to be included within the scope of the present invention.

What is claimed is:

1. A pharmaceutical composition which is stable and suitable for oral administration, comprising an effective amount of pravastatin or its pharmacetuically acceptable salts and a carrier, said carrier comprising at least one diluent and at least one lubricant to impart a pH between 6.5 and 8.5 to an aqueous dispersion of said composition.

2. The pharmaceutical composition according to claim 1 wherein the diluent may be water soluble, water dispersible, and mixtures thereof.

3. The pharmaceutical composition according to claim 2 wherein the water soluble diluent is selected from the group consisting of calcium carbonate, calcium phosphate, calcium hydrogen phosphate, tribasic calcium phosphate, calcium sulphate, compressible sugar, lactose, sucrose, sorbitol, mannitol, dextrates, dextrin, dextrose, maltodextrin, and mixtures thereof.

4. The pharmaceutical composition according to claim 2 wherein the water dispersible diluent is selected from the group consisting of cellulose, cellulosic derivatives, starch, starch derivatives, clay, clay minerals, and mixtures thereof.

5. The pharmaceutical composition according to claim 3 wherein the diluent is calcium carbonate.

6. The pharmaceutical composition according to claim 1 wherein the diluent comprises about 5% to about 95% by weight of said composition.

7. The pharmaceutical composition according to claim 6 wherein the diluent comprises about 15% to about 80% by weight of said composition.

8. The pharmaceutical composition according to claim 1 wherein the lubricant is selected from the group consisting of sodium stearyl fumarate, palmitic acid, calcium stearate, magnesium stearate, zinc stearate, talc, carnuba wax, silicon dioxide, hydrogenated vegetable oil, and mixtures thereof.

9. The pharmaceutical composition according to claim 8 wherein the lubricant is sodium stearyl fumarate.

10. The pharmaceutical composition according to claim 1 wherein the lubricant comprises about 0.1% to about 15% by weight of said composition.

11. The pharmaceutical composition according to claim 10 wherein the lubricant comprises about 0.2% to about 10% by weight of said composition.

12. The pharmaceutical composition according to claim 1 wherein the composition may further include adjuvants such as binders, disintegrants, surface active agents, and mixtures thereof.

13. The pharmaceutical composition according to claim 12 wherein the binder is selected from the group consisting of corn starch, polyvinyl alcohol, microcrystalline cellulose, polyvinyl pyrrolidine, modified corn starch, sugars, gums, methylcellulose, hydroxypropyl cellulose, and mixtures thereof.

14. The pharmaceutical composition according to claim 12 wherein the binder comprises about 0.1% to about 10% by weight of said composition.

15. The pharmaceutical composition according to claim 12 wherein the disintegrant is selected from the group consisting of croscarmellose sodium, starch, sodium starch glycolate, crospovidone, cross-linked carboxymethyl starch, magnesium aluminium silicate, polyacrylin potassium, and mixtures thereof.

16. The pharmaceutical composition according to claim 12 wherein the disintegrant comprises about 1% to about 10% by weight of said composition.

17. The pharmaceutical composition according to claim 12 wherein the surface active agent is selected from the group consisting of sodium lauryl sulphate, polyoxyethylene-polyoxypropylene copolymer, polysorbates, and mixtures thereof.

18. The pharmaceutical composition according to claim 12 wherein the surface active agent comprises about 1% to about 5% by weight of said composition.

19. The pharmaceutical composition according to claim 12 wherein the composition further comprises glidants, anti-adherents, colorants, or mixtures thereof.

20. The pharmaceutical composition according to claim 1 wherein the dosage form being formed into a physical form selected from the group consisting of pellets, beads, granules, tablets and capsules.

21. The pharmaceutical composition according to claim 20 wherein tablet dosage form further comprises coating with a fast dissolving film of a water soluble polymer.

22. The pharmaceutical composition according to claim 20 wherein the capsule shell is made of gelatin, hydroxypropyl methylcellulose or starch.

23. A dry process for the preparation of a pharmaceutical composition which is stable and suitable for oral administration, comprising an effective amount of pravastatin or its pharmaceutically acceptable salts and a carrier, said carrier comprising at least one diluent and at least one lubricant to impart a pH between 6.5 and 8.5 to an aqueous dispersion of said composition.

24. The process according to claim 23 wherein the dry process comprises direct compression or dry granulation.

25. The process according to claim 24 wherein dry granulation is performed using slugging or roller compaction.

26. The process according to claim 23 wherein the diluent may be water soluble, water dispersible, and mixtures thereof.

27. The process according to claim 26 wherein the water soluble diluent is selected from the group consisting of calcium carbonate, calcium phosphate, calcium hydrogen phosphate, tribasic calcium phosphate, calcium sulphate, compressible sugar, lactose, sucrose, sorbitol, mannitol, dextrates, dextrin, dextrose, maltodextrin, and mixtures thereof.

28. The process according to claim 26 wherein the water dispersible diluent is selected from the group consisting of cellulose, cellulosic derivatives, starch, starch derivatives, clay, clay minerals, and mixtures thereof.

29. The process according to claim 26 wherein the diluent is calcium carbonate.

30. The process according to claim 23 wherein the diluent comprises about 5% to about 95% by weight of the said composition.

31. The process according to claim 30 wherein the diluent comprises about 15% to about 80% by weight of the said composition.

32. The process according to claim 23 wherein the lubricant is selected from the group consisting of sodium stearyl fumarate, palmitic acid, calcium stearate, magnesium stearate, zinc stearate, talc, carnuba wax, silicon dioxide, hydrogenated vegetable oil, and mixtures thereof.

33. The process according to claim 32 wherein the lubricant is sodium stearyl fumarate.

34. The process according to claim 23 wherein the lubricant comprises about 0.1% to about 15% by weight of the said composition.

35. The process according to claim 34 wherein the lubricant comprises about 0.2% to about 10% by weight of the said composition.

36. The process according to claim 23 wherein the composition further comprises adjuvants such as binders, disintegrants, surface active agents, and mixtures thereof.

37. The process according to claim 36 wherein the binder is selected from the group consisting of corn starch, polyvinyl alcohol, microcrystalline cellulose, polyvinyl pyrrolidine, modified corn starch, sugars, gums, methylcellulose, hydroxypropyl cellulose, and mixtures thereof.

38. The process according to claim 36 wherein the binder comprises about 0.1% to about 10% by weight of the said composition.

39. The process according to claim 36 wherein the disintegrant is selected from the group consisting of croscarmellose sodium, starch, sodium starch glycolate, crospovidone, cross-linked carboxymethyl starch, magnesium aluminium silicate, polyacrylin potassium, and mixtures thereof.

40. The process according to claim 36 wherein the disintegrant comprises about 1% to about 10% by weight of the said composition.

41. The process according to claim 36 wherein the surface active agent is selected from the group consisting of sodium lauryl sulphate, polyoxyethylene-polyoxypropylene copolymer, polysorbates, and mixtures thereof.

42. The process according to claim 36 wherein the surface active agent comprises about 1% to about 5% by weight of the said composition.

43. The process according to claim 36 wherein the composition further comprises glidants, anti-adherents, colorants, or mixtures thereof.

44. The process according to claim 23 wherein the dosage form being formed into a physical form selected from the group consisting of pellets, beads, granules, tablets and capsules.

45. The process according to claim 44 wherein tablet dosage form further comprises coating with a fast-dissolving film of a water soluble polymer.

46. The process according to claim 44 wherein the capsule shell is made of gelatin, hydroxypropyl methylcellulose or starch.
